SEQUENCE LISTING (5 sequences)

SEQ ID NO: 1 - DNA, 31 bp, Artificial
  Chemically Synthesized
ggaattccatatgcatcaccatcaccatcac

SEQ ID NO: 2 - DNA, 41 bp, Artificial
  Chemically Synthesized
ccgctcgagttattacttaggtgggttaaggtagtcaatag

SEQ ID NO: 3 - DNA, 1383 bp, Escherichia coli
atgcatcaccatcaccatcacatgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaaatgtggaaacatccgatataaatctcatagttaatggcataattagaaagctaatcattttcttaagcatagagttatccttctaaagacttgttctttcagaaaaatgtttttccaattagtataaactctacaaattccctcttggtgtaaaattcaagatgtggaaattctaacttttttgaaatttaaaagcttatattttctaacttggcaaaagacttatccttagaaagagaagtgtacattgatttccaattaaaaatttgctggcattaaaaataagcacacttacataagcccccatacatagagtgggactcttggaatcaggagacaaagctaccacatgtggaaaggtactatgggtccatgtcattcaaaaaatgtgat

SEQ ID NO: 4 - DNA, 993 bp, Escherichia coli
atgcatcaccatcaccatcacatgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaataa

SEQ ID NO: 5 - protein, 329 aa, Escherichia coli
MHHHHHHMSAKSRTIGIIGAPFSKGQPRGGVEEGPTVLRKAGLLEKLKEQECDVKDYGDLPFADIPNDSPFQIVKNPRSVGKASEQLAGKVAQVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLGPSFTPATGTPVVGGLTYREGLYITEEIYKTGLLSGLDIMEVNPSLGKTPEEVTRTVNTAVAITLACFGLAREGNHKPIDYLNPPK

(19) United States
(12) Patent Application Publication
(10) Pub. No.: US 2010/0041101 A1
(43) Pub. Date: Feb. 18, 2010

(54) EXPRESSION SYSTEM FOR RECOMBINANT HUMAN ARGINASE I

(75) Inventors: Yu Liang Huang, Shanghai (CN); Zhong Shu Xian, Shanghai (CN)

Correspondence Address: EAGLE IP LIMITED, 13/F, Bright Way Tower, 33 Mong Kok Road, Kowloon, HK

(73) Assignee: BIO-CANCER TREATMENT INTERNATIONAL LIMITED, Shatin (HK)

(21) Appl. No.: 12/514,585

(22) PCT Filed: Nov. 20, 2007
(86) PCT No.: PCT/US07/85319

§ 371 Date: May 13, 2009

(30) Foreign Application Priority Data
Dec. 12, 2006 (US) 11609902

(51) Int. Cl. C12P 21/02; C12N 15/74; C12N 1/21

(52) U.S. Cl. 435/69.1; 435/320.1; 435/252.33

(57) ABSTRACT

A novel recombinant protein expression system is provided for improving expression of recombinant human arginase I. The system contains an isolated and purified nucleic acid molecule for constructing plasmid and E. coli strain in order to improve the expression of recombinant human arginase I. In another aspect of the present invention, a method is provided for producing an isolated E. coli strain in expressing said arginase.

### FIELD OF INVENTION

The present invention is related to the cloning of human arginase I. In particular, the present invention is related to nucleic acid molecules and plasmids that correspond to said human arginase I. The present invention also relates to a strain of E. coli for expression of said recombinant protein of human arginase I. The present invention also relates to a method of producing a recombinant protein.

### BACKGROUND OF INVENTION

Recombinant process uses genetically engineered organisms to produce useful proteins for medical use. Some examples of product made by recombinant process are insulin, growth hormones and vaccines. Large amounts of the protein can be produced in a factory with vats of the genetically engineered bacteria. In recombinant process, organism most commonly used is Escherichia coli.

Bacteria physiology and genetics are probably far better understood than for any other living organism However, the success or failure of a process often depends on the survival rate of the genetically engineered bacteria and the recombinant DNA which carries the essential information for making the final product. Poorly constructed plasmid may become unable to produce meaningful amount of product yet lower the survival rate of the genetically engineered bacteria. There are also risks of producing contaminations hard to eliminate and worsen the quality of the final product.

### SUMMARY OF INVENTION

In view of the foregoing background, it is an object of the present invention to provide a better genetically engineered bacteria in producing human arginase I so as to maximize output of producing said arginase, making the method safe and efficient for the production of pharmaceutical GMP grade material.

Accordingly, the present invention, in one aspect, is an isolated and purified nucleic acid molecule for the expression of recombinant human arginase I.

A preferred embodiment of the present invention is the use of the aforesaid nucleic acid molecule in constructing a plasmid for expression of recombinant human arginase I.

A further aspect of the invention is the use of the aforesaid plasmid in constructing an isolated strain of Escherichia coli for the production of recombinant human arginase I.

### BRIEF DESCRIPTION OF FIGURES

FIG. 1 shows the agarose electrophoretic analysis of plasmid extraction of pET30(+)/ARGC from transformed competent DH5(α) E. coli cells. Extracted pET30(+)/ARGC was digested with the restrictive enzymes NdeI and XhoI. Expected fragment sizes of 1.4 kb and 5 kb were shown. Lane M: λ DNA/EcoRI+HindIII Marker (MBI); Lane 1: pET30a(+)/ARGC double-digested with NdeI and XhoI; Lane 2: Undigested pET30a(+)/ARGC.

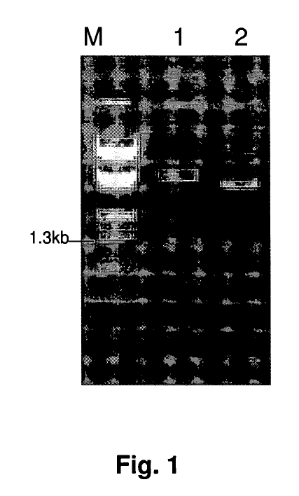

FIG. 2 shows the inserted nucleotide sequence of the recombinant pET30(+)/ARGC, containing 1,383 nucleic acids.

FIG. 3 shows the agarose electrophoretic analysis of plasmid extraction of pET30(+)/ARGM from transformed competent DH5(α) E. coli cells. Extracted pET30(+)/ARGM was digested with the restrictive enzymes NdeI and XhoI. Expected fragment sizes of 1 kb and 5 kb were shown. Lane M: λ DNA/EcoRI+HindIII Marker (MBI); Lane 1: pET30a(+)/ARGM double-digested with NdeI and XhoI; Lane 2: Undigested pET30a(+)/ARGM.

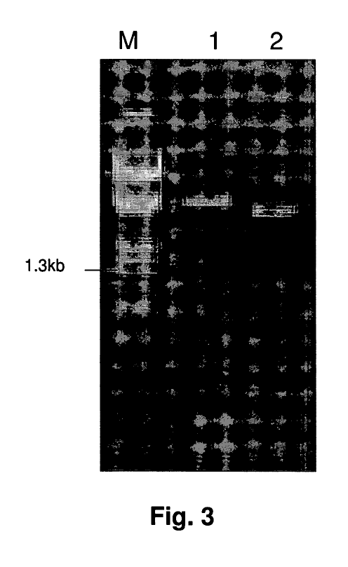

FIG. 4 shows the inserted nucleotide sequence of the recombinant pET30(+)/ARGM, containing 993 nucleic acids, including 2 sets of stop codon TAA.

FIG. 5 shows the amino acid sequence deduced from the nucleotide sequence of the 993 nucleic acids coding region of pET30a(+)/ARGM. The expressed human arginase I protein is a protein of 322 amino acid residues plus an initiation methionine and a tag of 6 histidines, or 329 amino acid residues in total.

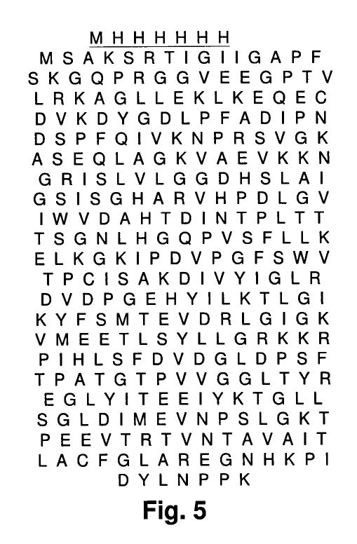

FIG. 6 shows the SDS-PAGE analysis of the pAED-4/ARGC expressed by BL21(DE3). Lane M: low molecular weight protein marker; Lane 1: recombinant human arginase I without IPTG induction; Lane 2: 1 h after induction; Lane 3: 2 h after induction; Lane 4: 3 h after induction; Lane 5: 4 h after induction; Lane 6: 5 h after induction.

FIG. 7 shows the SDS-PAGE analysis of the pET30a(+)/ARGC expressed by BL21(DE3). Lane M: low molecular weight protein marker; Lane 1: recombinant human arginase I without IPTG induction; Lane 2: 1 h after induction; Lane 3: 2 h after induction; Lane 4: 3 h after induction; Lane 5: 4 h after induction; Lane 6: 5 h after induction.

FIG. 8 shows the SDS-PAGE analysis of the pET30a(+)/ARGM expressed by BL21(DE3). Lane M: low molecular weight protein marker; Lane P: pure human arginae I; Lane 1: recombinant human arginase I without IPTG induction; Lane 2: 1 h after induction; Lane 3: 2 h after induction; Lane 4: 3 h after induction; Lane 5: 4 h after induction; Lane 6: 5 h after induction.

### DETAILED DESCRIPTION OF THE PREFERRED EMBODIMENTS

### Example 1

### Construction of the pET30a(+)/ARGC Plasmid

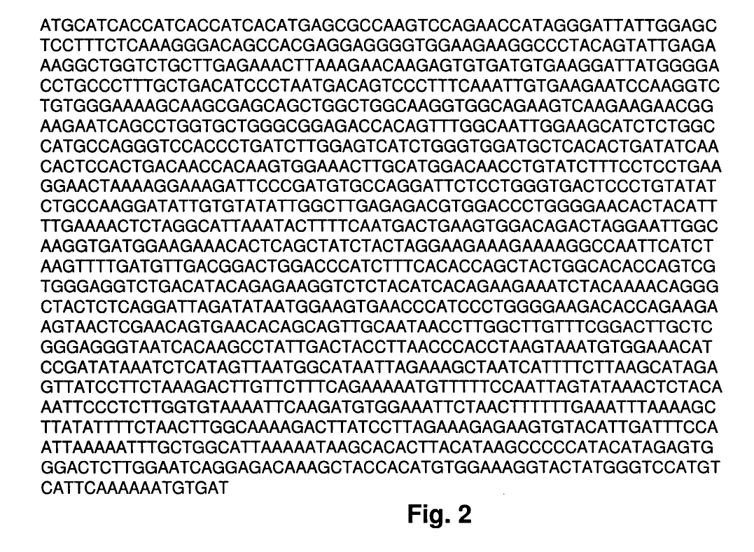

The plasmid pET30a(+)/ARGC plasmid was prepared using experimental techniques common in the field of gene cloning. First, both pAED-4/ARGC plasmid and pET30a(+) plasmid were independently subjected to overnight digestion at 37° C. with the restrictive enzymes NdeI and XhoI. The digested fragments were then mixed with T4 DNA ligase at 16° C. overnight. The ligated plasmid was transformed into competent DH5(α) E. coli cells. Selection was performed on LB plates comprising 30 μg/mL kanamycin. Single colonies were picked and cultured. The ligated plasmid was extracted and confirmed by digestion using the restrictive enzymes NdeI and XhoI at 37° C. for 1 hour and electrophoresis. Ultimately, the ligated and extracted plasmid contained a pET30(+) backbone and the human arginase gene (containing non-coding sequence) was named pET30(+)/ARGC. The nucleic acid sequence was confirmed by Invitrogen Biotechnology Co., Ltd (Shanghai). As shown in FIG. 2, it was identical with the theorized sequence, consisting of 1,383 nucleic acids.

### Example 2

### Expression of the pET30a(+)/ARGC Plasmid

The constructed pET30a(+)/ARGC was used to transform competent BL21 (DE3) E. coli cells on LB plates containing 30 μg/mL kanamycin. After 12 hours growth time, single colonies were picked and transferred into 50 mL LB media. The cells were fermented at 37° C. at 250rpm. At OD₆₀₀ 0.6 to 0.8, IPTG was added to a concentration of 0.4 mM to induce expression. SDS-PAGE is used to test the expression level.

### Example 3

### Construction of pET30a(+)/ARGM Plasmid

Two primers (SEQ ID NO. 1 and 2) were designed for the construction of pET30a(+)/ARGM plasmid using the restrictive enzymes NdeI and XhoI, as follows:

1-F:
5′-GGAATTCCATATGCATCACCATCACCATCAC-3′
2-R:
5′-CCGCTCGAGTTATTACTTAGGTGGGTTAAGGTAGTCAATAG-3

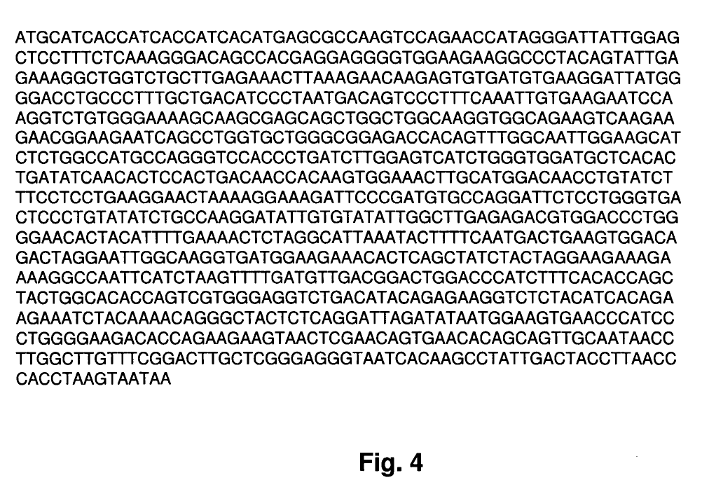

The plasmid pET30a(+)/ARGM was prepared using experimental techniques common in the field of gene cloning. First, amplify pAED-4/ARGC plasmid by Polymerase Chain Reaction (PCR) using pAED-4/ARGC plasmid as the template. The amplified gene fragments and pET30a(+) plasmid were independently subjected to overnight digestion at 37° C. with the restrictive enzymes NdeI and XhoI. The digested fragments were then mixed with T4 DNA ligase at 16° C. overnight. The ligated plasmid was transformed into competent DH5(α) E. coli cells. Selection was performed on LB plates comprising 30 μg/mL kanamycin. Single colonies were picked and cultured. The ligated plasmid was extracted and confirmed by digestion using the restrictive enzymes NdeI and XhoI at 37° C. for 1 hour and electrophoresis. Ultimately, the ligated and extracted plasmid contained a pET30(+) backbone and the human arginase gene (without the non-coding sequence), was named pET30(a)/ARGM. The nucleic acid sequence was sent to and confirmed by Invitrogen Biotechnology Co., Ltd (Shanghai). As shown in FIG. 4, it was identical with the theorized sequence, consisting of 993 nucleic acids.

### Example 4

### Expression of the pET30a(+)/ARGM Plasmid

The constructed pET30a(+)/ARGM was used to transform competent BL21 (DE3) E. coli cells on LB plates containing 30 μg/mL kanamycin. After 12 hours growth time, single colonies were picked and transferred into 50 mL LB media. The cells were fermented at 37° C. at 250 rpm. At OD₆₀₀ 0.6 to 0.8, IPTG was added to a concentration of 0.4 mM to induce expression. SDS-PAGE is used to test the expression level.

### Example 5

### Comparison of Expression Level among the Human Arginase I Expressed in BL21(DE3) E. coli

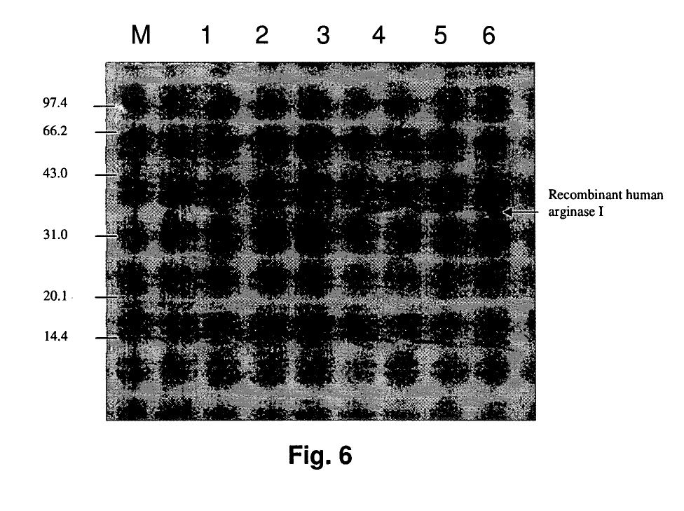

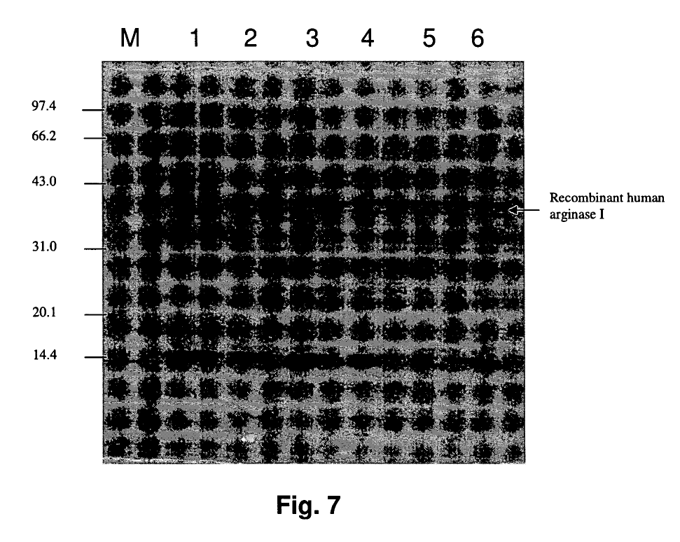

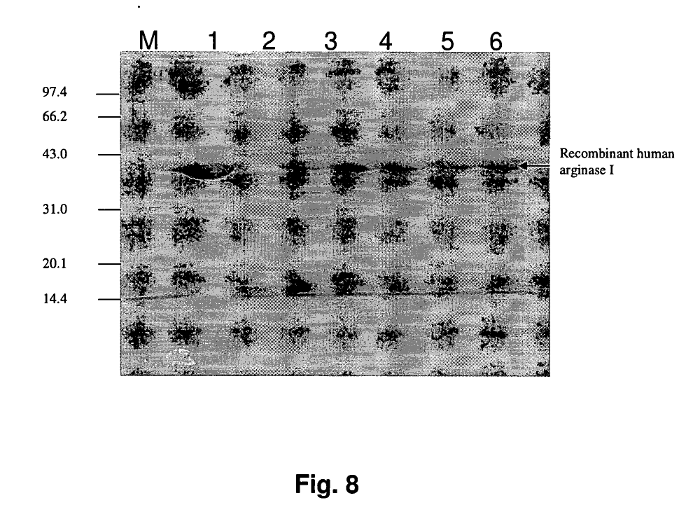

FIG. 6 shows the expression level of human arginase from BL21(DE3) E. coli cells transformed with pAED-4/ARGC. It is apparent that the impurity is high, while the expression level is low. FIG. 7 shows the expression level of recombinant human arginase from BL21(DE3) E. coli cells transformed with pET30a(+)/ARGC. It is apparent that the content contains less purity as compared to cells transformed with pAED-4/ARGC. Although the expression level is slightly higher than those expressed by pAED-4/ARGC as in FIG. 6, the yield of expressed human arginase I is still low. FIG. 8 shows the expression level of human arginase from BL21(DE3) E. coli cells transformed with pET30a(+)/ARGM. It can be seen that the content is the most pure among the three plasmids, and the expression level is the highest.

### Example 6

### Comparison of Plasmid Stability among the Human Arginase I Expressed in BL21(DE3) E. coli

Table 1, 2 and 3 show the comparison of physiological characteristics of E. coli cells transformed with pAED-4/ARGC, pET30a(+)/ARGC and pET30a(+)/ARGM, in terms of plasmid stability. Initially, E. coli cells transformed with pAED-4/ARGC and pET30a(+)/ARGC showed normal growth rate and kanamycin resistance. After 4 months of storage in glycerol at −80° C., no colony was detected until the dilution fold was decreased to 10e4-10e5, and no gene expression was detected from the fermentation broth.

E. coli cells transformed with pET30a(+)/ARGM initially showed normal kanamycin resistance at the dilution fold of 10e9-10e10. Also, expression level was found to be 15% to 25%, which was much higher than that of pAED-4/ARGC and pET30a(+)/ARGC transformed cells. After 6 months of storage in glycerol at −80° C., pET30a(+)/ARGM transformed cells retained the normal level of kanamycin resistance, and expression level was much higher than that of pAED-4/ARGC and pET30a(+)/ARGC transformed cells after 4 months −80° C. storage.

TABLE 1
physiological properties of pAED-4/ARGC
transformed BL21(DE3)

| | Dilution fold at | Gene expression induced by IPTG, |
|---|---|---|
| Time | detecting colonies | extrapolated from SDS PAGE |
| T₀ | 10e9-10e10 | ~7% |
| T_{4 months} | 10e4-10e5 | 0% |

TABLE 2
physiological properties of pET30a(+)/ARGC
transformed BL21(DE3)

| | Dilution fold at | Gene expression induced by IPTG, |
|---|---|---|
| Time | detecting colonies | extrapolated from SDS PAGE |
| T₀ | 10e9-10e10 | ~7% |
| T_{4 months} | 10e4-10e5 | 0% |

TABLE 3
physiological properties of pET30a(+)/ARGM
transformed BL21(DE3)

| | Dilution fold at | Gene expression induced by IPTG, |
|---|---|---|
| Time | detecting colonies | extrapolated from SDS PAGE |
| T₀ | 10e9-10e10 | 15%-25% |
| T_{6 months} | 10e9-10e10 | 15%-25% |

The preferred embodiments of the present invention are thus fully described. Although the description referred to particular embodiments, it will be clear to one skilled in the art that the present invention may be practiced with variation of these specific details. Hence, this invention should not be construed as limited to the embodiments set forth herein.

For example, although the present invention referred to using pET30a(+) vector from Novagen, a person skilled in the art will appreciate that other vectors may be employed, such as pTrcHis (Invitrogen), pGEX (Amersham Biosciences), pBAD (Invitrogen), pRSET (Invitrogen), pBV220, and pQE (Qiagen).

A person skilled in the art will also appreciate that although the present invention referred to using a lac promoter, a person skilled in the art will appreciate that other promoters may be used, such as tryptophan promoter, Trc promoter, Tac promoter, araBAD promoter, T7 promoter, T5 promoter, and temperature induced promoter.

Furthermore, a person skilled in the art will also appreciate that although the present invention referred to using BL21(DE3) as host, other expression systems may be employed, such as TOP10, M15, and DH5a E. coli.

The present invention has been described using the encoding region of human arginase I, which consists of 990 bp including the final TAA which transcribes into the stop codon UAA. The most preferred embodiment of the present invention uses an encoding region of human arginase I consisting of 993 bp, which an additional set of TAA is included to further ensure the expression of the terminal signal.

What is claimed is:

1. An isolated and purified nucleic acid molecule for expression of recombinant human arginase I, wherein said nucleic acid molecule comprises the encoding sequence of human arginase I and a predetermined promoter sequence operably linked thereto for stimulating the expression of said human arginase I in a predetermined expression system, and wherein said nucleic acid sequence excludes non-coding sequences of the human arginase I mRNA.

2. The isolated and purified nucleic acid molecule according to claim 1, wherein said nucleic acid molecule further comprises a nucleic acid sequence encoding a plurality of histidines.

3. The isolated and purified nucleic acid molecule according to claim 2, wherein said nucleic acid sequence encodes at least six histidines.

4. A plasmid for expression of recombinant human arginase I, wherein said plasmid comprises the encoding sequence of human arginase I and a predetermined promoter sequence operably linked thereto for stimulating the expression of said human arginase I in a predetermined expression system, and wherein said plasmid excludes non-coding sequences of the human arginase I mRNA.

5. The plasmid according to claim 4, wherein said plasmid comprises a nucleic acid sequence encoding a plurality of histidines.

6. The plasmid according to claim 5, wherein said nucleic acid sequence encodes at least six histidines.

7. The plasmid according to claim 4, wherein said promoter sequence encodes a lac operon operably linked to said encoding sequence of human arginase I.

8. An isolated E. coli strain for expression of recombinant human arginase I, wherein said E. coli comprises a nucleic acid molecule comprising the encoding sequence of human arginase I and a predetermined promoter sequence operably linked thereto for stimulating the expression of said human arginase I in a predetermined expression system, and wherein said nucleic acid sequence excludes non-coding sequences of the human arginase I mRNA.

9. An isolated E. coli strain according to claim 8, wherein said nucleic acid molecule comprises a nucleic acid sequence encoding a plurality of histidines.

10. An isolated E. coli strain according to claim 9, wherein said nucleic acid sequence encodes at least six histidines.

11. An isolated E. coli strain according to claim 8, wherein said nucleic acid molecule comprises a lac operon sequence downstream of a T7 promoter, operably linked to said nucleic acid molecule.

12. A method of producing recombinant protein comprising:
a) constructing a recombinant E. coli strain according to claim 8;
b) fermenting said recombinant E. coli cells using fed-batch fermentation;
c) inducing said recombinant E. coli cells to stimulate expression of said recombinant protein; and
d) purifying said recombinant protein from the product of said fermentation.

13. The method according to claim 12 wherein said human arginase I has at least six histidines linked thereof, and said purifying step comprises affinity chromatography in a chelating column.
